(19) United States
(12) Patent Application Publication
(10) Pub. No.: US 2010/0233752 A1
(43) Pub. Date: Sep. 16, 2010

(54) METHOD FOR DIAGNOSIS AND MONITORING OF DISEASE ACTIVITY AND RESPONSE TO TREATMENT IN SYSTEMIC LUPUS ERYTHEMATOSUS (SLE) AND OTHER AUTOIMMUNE DISEASES

(75) Inventors: Joseph M. Ahearn, Sewickley, PA (US); Edward L. Erickson, Pipersville, PA (US); Douglas M. Hawkins, Edina, MN (US); Susan M. Manzi, Wexford, PA (US); Thomas Mercolino, Chapel Hill, NC (US)

Correspondence Address: COOLEY LLP;ATTN: Patent Group, Suite 1100, 777 - 6th Street, NW, WASHINGTON, DC, 20001, US

(73) Assignees: CYPRESS BIOSCIENCE, INC., San Diego, CA (US); UNIVERSITY OF PITTSBURGH, Pittsburgh, PA (US)

(21) Appl. No.: 12/581,057

(22) Filed: Oct. 16, 2009

Related U.S. Application Data
(60) Provisional application No. 61/105,865, filed on Oct. 16, 2008.
(60) Provisional application No. 61/143,225, filed on Jan. 8, 2009.

(51) Int. Cl. C12Q 1/02

(52) U.S. Cl. 435/29

(57) ABSTRACT

The present invention provides methods of diagnosing and monitoring systemic lupus erythematosus and drug-induced lupus erythematosus by measuring cell-based complement activation products in a subject's blood. In particular, the invention describes a diagnostic method employing the measurement of multiple complement activation products, such as C3d and C4d, on the surfaces of red blood cells, white blood cells, and platelets. Kits and automated systems for performing the methods of the invention are also disclosed.

### CROSS-REFERENCE TO RELATED APPLICATIONS

This application claims the benefit of priority of U.S. Provisional Application No. 61/105,865, filed Oct. 16, 2008, and U.S. Provisional Application No. 61/143,225, filed Jan. 8, 2009, both of which are herein incorporated by reference in their entireties.

### BACKGROUND OF THE INVENTION

Systemic Lupus Erythematosus (SLE) is an autoimmune disease, characterized by the production of unusual autoantibodies in the blood. These autoantibodies bind to their respective antigens, forming immune complexes which circulate and eventually deposit in tissues. This immune complex deposition causes chronic inflammation and tissue damage.

The precise reason for the abnormal autoimmunity that causes lupus is not known. Inherited genes, viruses, ultraviolet light, and drugs may all play some role. Genetic factors increase the tendency of developing autoimmune diseases, and autoimmune diseases such as lupus, rheumatoid arthritis, and immune thyroid disorders are more common among relatives of patients with lupus than the general population. Some scientists believe that the immune system in lupus is more easily stimulated by external factors like viruses or ultraviolet light. Sometimes, symptoms of lupus can be precipitated or aggravated by only a brief period of sun exposure.

Since patients with SLE can have a wide variety of symptoms and different combinations of organ involvement, no single test establishes the diagnosis of SLE. To help doctors improve the accuracy of diagnosis of SLE, eleven criteria were established by the American Rheumatism Association. These eleven criteria are closely related to the variety of symptoms observed in patients with SLE. When a person has four or more of these criteria, the diagnosis of SLE is strongly suggested. However, some patients suspected of having SLE may never develop enough criteria for a definite diagnosis. Other patients accumulate enough criteria only after months or years of observation. Nevertheless, the diagnosis of SLE may be made in some settings in patients with only a few of these classical criteria. Of these patients, a number may later develop other criteria, but many never do. The eleven criteria conventionally used for diagnosing SLE are:
1—malar over the cheeks of the face or “butterfly” rash
2—discoid skin rash: patchy redness that can cause scarring
3—photosensitivity: skin rash in reaction to sunlight exposure
4—mucus membrane ulcers: ulcers of the lining of the mouth, nose or throat
5—arthritis: two or more swollen, tender joints of the extremities
6—pleuritis/pericarditis: inflammation of the lining tissue around the heart or lungs, usually associated with chest pain with breathing
7—kidney abnormalities: abnormal amounts of urine protein or clumps of cellular elements called casts
8—brain irritation: manifested by seizures (convulsions) and/or psychosis
9—blood count abnormalities: low counts of white or red blood cells, or platelets
10—immunologic disorder: abnormal immune tests include anti-dsDNA or anti-Sm (Smith) antibodies, false positive blood tests for syphilis, anticardiolipin antibodies, lupus anticoagulant, or positive LE prep test, and
11—antinuclear antibody: positive ANA antibody testing

Although the criteria serve as useful reminders of those features that distinguish lupus from other related autoimmune diseases, they are unavoidably fallible. Determining the presence or absence of the criteria often requires interpretation. If liberal standards are applied for determining the presence or absence of a sign or symptom, one could easily diagnose a patient as having lupus when in fact they do not. Similarly, the range of clinical manifestations in SLE is much greater than that described by the eleven criteria and each manifestation can vary in the level of activity and severity from one patient to another. To further complicate a difficult diagnosis, symptoms of SLE continually evolve over the course of the disease. New symptoms in previously unaffected organs can develop over time. Because conventionally there is no definitive test for lupus, it is often misdiagnosed.

Monitoring disease activity is also problematic in caring for patients with lupus. Lupus progresses in a series of flares, or periods of acute illness, followed by remissions. The symptoms of a flare, which vary considerably between patients and even within the same patient, include malaise, fever, symmetric joint pain, and photosensitivity (development of rashes after brief sun exposure). Other symptoms of lupus include hair loss, ulcers of mucous membranes and inflammation of the lining of the heart and lungs which leads to chest pain.

Red blood cells, platelets and white blood cells can be targeted in lupus, resulting in anemia and bleeding problems. More seriously, immune complex deposition and chronic inflammation in the blood vessels can lead to kidney involvement and occasionally failure requiring dialysis or kidney transplantation. Since the blood vessel is a major target of the autoimmune response in lupus, premature strokes and heart disease are not uncommon. Over time, however, these flares can lead to irreversible organ damage. In order to minimize such damage, earlier and more accurate detection of disease flares would not only expedite appropriate treatment, but would reduce the frequency of unnecessary interventions. From an investigative standpoint, the ability to uniformly describe the “extent of inflammation” or activity of disease in individual organ systems or as a general measure is an invaluable research tool. Furthermore, a measure of disease activity can be used as a response variable in a therapeutic trial.

Two of the most commonly used instruments are the Systemic Lupus Disease Activity Index (SLEDAI) (Bombardier, C., D. D. Gladman, et al. (1992), Arth Rheum 35: 630-40), and the Systemic Lupus Activity Measure (SLAM) (Liang, M. H., S. A. Socher, et al. (1989), Arth Rheum 32: 1107-18). The SLEDAI includes 24 items representing 9 organ systems. The variables are obtained by history, physical examination and laboratory assessment. Each item is weighted from 1 to 8 based on the significance of the organ involved. For example, mouth ulcers are scored as 2, while seizures are scored as 8. The laboratory parameters that are included in the SLEDAI include white blood cell count, platelet count, urinalysis, serum C3, C4 and anti-double-stranded DNA (anti-dsDNA). The total maximum score is 105. The SLAM includes 32 items representing 11 organ systems. The items are scored not only as present/absent, but graded on a scale of 1 to 3 based on severity. The total possible score for the SLAM is 86. Both the SLEDAI and the SLAM have been shown to be valid, reliable, and sensitive to change over time (Liang, M. H., S. A. Socher, et al. (1989), Arth Rheum 32:1107-18), and are widely used in research protocols and clinical trials. These indices are particularly useful for examining the value of newly proposed serologic or inflammatory markers of disease activity in SLE.

Despite the obvious utility of these instruments, there are drawbacks. First, there is not always complete agreement between the SLAM and the SLEDAI in the same set of patients. There are several possible reasons for these discrepancies. Unlike the SLEDAI, the SLAM includes symptoms such as fatigue and fever, which may or may not be considered attributable to active SLE; this activity index relies on physician interpretation. In addition, the SLEDAI does not capture mild degrees of activity in some organ systems and does not have descriptors for several types of activity such as hemolytic anemia. For these and other reasons, most studies incorporate more than one measure of disease activity.

In addition to SLE, lupus can manifest in response to chronic exposure to several pharmacological agents. Such drug-induced lupus erythematosus (DILE) is a syndrome sharing symptoms and laboratory findings with SLE (Sarzi-Puttini et al. (2005), Autoimmunity 38(7):507-18). DILE can arise months to years after exposure to drugs prescribed to treat a variety of medical conditions. Additionally, biologics (e.g., genetically-engineered proteins) are associated with induction of DILE symptoms (Haraoui, B and Keystone, E (2006), Curr Opin Rheumatol 18(1):96-100) and many small molecule drugs and biologics will induce flares of disease symptoms in SLE patients. Taken together, the breadth of pharmacologic agents associated with DILE strongly suggests that DILE would be a likely side effect of pharmacologic agents in development and as yet to be developed. Thus, early recognition of DILE is important because it usually reverts within a few weeks after stopping the drug. Further, early recognition of DILE in clinical trial subjects may be a means for avoiding severe adverse events that mitigate against eventual drug approval.

In an active disease state with severe inflammation, i.e., a flare, the expectation is that there will be a significant increase of anti-dsDNA and a significant decrease/consumption of serum C3 and C4 indicating serious activation of the complement system. If there is a positive response to therapy, it is expected that these levels will return to normal. Furthermore, it is expected that the activity indices (SLAM—SLE Activity Measure, SLEDAI—SLE Disease Activity Index and the BILAG—British Isles Lupus Assessment Group index) will increase and decrease with response or lack of response to therapy. It is widely accepted within the rheumatology community that the anti-dsDNA test is better for monitoring response to therapy than serum C3 and C4. However, as noted in the literature, a significant percentage of SLE patients will never test positive for anti-dsDNA (e.g., 25-35% depending on the study).

In general, the complement system consists of a complex network of more than 30 functionally linked proteins that interact in a highly regulated manner to provide many of the effector functions of humoral immunity and inflammation, thereby serving as the major defense mechanism against bacterial and fungal infections. This system of proteins acts against invasion by foreign organisms via three distinct pathways: the classical pathway (in the presence of antibody), the alternative pathway (in the absence of antibody), and the lectin pathway. Once activated, the proteins within each pathway form a cascade involving sequential self-assembly into multimolecular complexes that perform various functions intended to eradicate the foreign antigens that initiated the response.

The classical pathway is usually triggered by an antibody bound to a foreign particle. It consists of several components that are specific to the classical pathway and designated C1, C4, C2, (in that order in the pathway). In the classical pathway, the first component C1q is bound to an antigen-antibody complex, activating the pathway. This event is followed by sequential activation of the two serine proteases C1r and C1s. Activated C1s has two substrates, the final two proteins of the classical pathway, namely C4 and C2. Protein C4 is cleaved into C4a and C4b. Protein C2 is cleaved to form C2a and C2b. Fragments C4b and C2a assemble to form C4b2a, which cleaves protein C3 into C3a and C3b, completing activation of the classical pathway.

Fragments C4b and C3b are subject to further degradation by Factor I. This factor cleaves C4b to generate C4d and also cleaves C3b, to generate iC3b followed by C3d. Thus, activation of the classical pathway of complement can lead to deposition of a number of fragments, including C4d and iC3b on immune complexes or other activating surfaces. These fragments are ligands for complement receptor type 1 (CR1) expressed on the surface of erythrocytes or red blood cells.

There have been inconsistent reports regarding complement proteins and SLE. One manifestation that has been reported in patients having SLE is a diminished expression of the complement receptor CR1 on erythrocytes (E-CR1) as compared to normal individuals (see, e.g., Ross et al. (1985), J. Immunol., Vol. 135: 2005; Corvetta et al. (1991), J. Rheumatol., Vol. 18: 1021). Iida et al. (J. Exp. Med. 155, 1427 (1982)) noted that the number of CR1 receptors on erythrocytes varied inversely with disease activity, with lower numbers occurring during periods of most severe manifestations of SLE, and higher numbers being observed during periods of remission in the same patients. Other studies seem to show there is no correlation.

Thus, the desired attributes of an effective and actionable monitoring test or panel of tests for SLE or DILE include the ability to gauge disease activity, monitor and/or predict response to treatments, correlate with favorable outcomes and monitor and/or predict the onset of flares. Because the diagnosis of SLE evolves over time, lupus specialists heretofore have relied largely on various indices such as SLEDAI or SLAM. If these scales are used to evaluate a new modality or if they are inherently “noisy”, then the new modality may not look useful or effective even though in reality it does reflect the actual biological and physiological status of the disease. Therefore, there is a need in the art to develop improved methods to effectively diagnose SLE and monitor the therapeutic response in SLE patients as well as detect the development of DILE.

### SUMMARY OF THE INVENTION

The present invention is based, in part, on the discovery that detection of cell-bound complement activation products (CB-CAPS) in blood samples can be used alone or in various combinations to provide a more sensitive and specific method of diagnosing and monitoring Systemic Lupus Erythematosus (SLE) in a subject. Accordingly, the present invention provides a method for diagnosing SLE in a subject by determining the level of at least one CB-CAP in a sample from the subject. In one embodiment, the method comprises determining the level of a first biomarker in a sample from a subject, wherein if the level of the first biomarker is within a first predetermined level, the subject is diagnosed with SLE. If the level of the first biomarker is outside the predetermined level, then the level of a second biomarker is determined, and if the level of the second biomarker is within a second predetermined level, the subject is diagnosed with SLE. Generally, the first and second biomarkers are different. In certain embodiments, the first and second biomarkers are selected from the group consisting of BC4d, EC4d, PC4d and TC4d. In one embodiment, the first biomarker is EC4d and the second biomarker is BC4d.

In another embodiment, the method of diagnosing SLE further comprises determining the level of a third biomarker if the level of the second biomarker is outside of the second predetermined level, wherein if the level of the third biomarker is within a third predetermined level, the subject is diagnosed with SLE. The third biomarker is typically different than the first two biomarkers. In one embodiment, the third biomarker is EC4d, ECR1, or a combination thereof. In some embodiments, the first biomarker is BC4d or TC4d, the second biomarker is PC4d, and the third biomarker is EC4d, ECR1, or a combination thereof. In other embodiments, the first biomarker is PC4d, the second biomarker is TC4d, and the third biomarker is EC4d, ECR1, or a combination thereof.

In another embodiment of the invention, a method for diagnosing SLE comprises determining the level of EC4d and EC3d in a sample from a subject and determining whether the subject has SLE based on the level of EC4d and EC3d. In some embodiments, the method further comprises determining the level of ECR1 and determining whether the subject has SLE based on the level of EC4d, EC3d, and ECR1. In other embodiments, the method further comprises determining the level of at least one additional biomarker. The additional biomarker can include PC4d, TC3d, TC4d, BC3d, and MC4d.

In some embodiments, the methods of the invention are performed in subjects that are negative for conventional tests, such as the antinuclear antibody test or anti-double stranded DNA test.

The present invention also includes a method for facilitating diagnosis of SLE. In one embodiment, the method comprises determining the level of a first biomarker in a sample from a subject, wherein if the level of the first biomarker is outside a predetermined level, then the level of a second biomarker is determined, and providing the level of the first biomarker and the second biomarker to an entity for diagnosis of SLE. Generally, the first and second biomarkers are different. In certain embodiments, the first and second biomarkers are selected from the group consisting of BC4d, EC4d, PC4d and TC4d. In one embodiment, the first biomarker is EC4d and the second biomarker is BC4d.

In another embodiment, the method further comprises determining the level of a third biomarker if the level of the second biomarker is outside of the second predetermined level. The third biomarker is typically different from the first and second biomarker and can, in some embodiments, be EC4d, ECR1, or a combination thereof. In some embodiments, the first biomarker is BC4d or TC4d, the second biomarker is PC4d, and the third biomarker is EC4d, ECR1, or a combination thereof. In other embodiments, the first biomarker is PC4d, the second biomarker is TC4d, and the third biomarker is EC4d, ECR1, or a combination thereof.

The present invention also encompasses a method of detecting the development of drug-induced lupus erythematosus (DILE) in a subject who is being treated with a pharmacological agent. In one embodiment, the method comprises determining the level of at least one biomarker in a sample from the subject and determining whether the subject has DILE based on the level of the at least one biomarker. The biomarker can be selected from the group consisting of BC4d, EC4d, PC4d, TC4d, ECR1, MC4d, TC3d, BC3d, and combinations thereof. In some embodiments, the subject is being treated with an agent that modulates the activity of the immune system, such as a biologic that affects the expression or activity of one or more cytokines.

The present invention also provides kits and combinations of tests useful for diagnosing SLE according to the methods of the invention described herein. In one embodiment, the combination of tests comprises a first test for the level of EC4d, a second test for the level of EC3d, and a third test for the level of ECR1. In another embodiment, the combination further comprises at least one additional test for determining the level of an additional biomarker, such as PC4d, TC3d, TC4d, BC3d, and MC4d.

A collection of results of the various levels of the biomarkers in a readable format for diagnosing SLE is also encompassed by the present invention. In one embodiment, the collection of results comprises the level of EC4d, the level of EC3d, and the level of ECR1.

### DETAILED DESCRIPTION OF THE INVENTION

The present invention provides methods for the diagnosis and monitoring of disease activity and response to treatment in Systemic Lupus Erythematosus (SLE) and other autoimmune diseases using panels of biomarkers. In particular, the present invention includes the use of cell-bound complement activation products (CB-CAPS) as biomarkers to facilitate the diagnosis and monitoring of SLE in a subject. This panel of biomarkers can be used alone or in combination with traditional diagnostic assays (e.g., serum C3 levels, serum C4 levels, and anti-double-stranded DNA antibodies) to enhance the specificity and sensitivity of SLE diagnosis and disease monitoring.

Conventionally, a decrease in serum C3 and serum C4 levels has been used as a diagnostic marker for SLE because a decrease in these complement proteins was indicative of severe activation of the complement system. However, there are conflicting reports in the literature on the usefulness of these serum measurements of complement proteins to assess SLE status because serum C3 and C4 levels rarely quantitatively correlate with disease severity and there is a large variation of serum C3 and C4 levels in SLE patients that overlap with healthy subjects (Manzi et al. (2004) Lupus, Vol. 13:298-303). The presence of anti-dsDNA antibodies has also been widely accepted as an appropriate test for diagnosing SLE and monitoring disease severity. However, there are a subset of SLE patients (approximately 25-35%) that never test positive for anti-dsDNA antibodies. Thus, traditional measures for diagnosing and monitoring SLE in a subject lack accuracy and sensitivity, and improved methods of diagnosis using additional biomarkers are needed.

The present invention addresses this need by providing a panel of biomarkers that can be used alone or in combination with the traditional assays to facilitate the diagnosis and monitoring of SLE. As used herein, “biomarker” or “marker” refers to a biochemical molecule, macromolecule, or metabolite that can be measured in a biological sample from a subject that identifies a particular condition (e.g., SLE or DILE) or status of a condition (e.g., level of disease progression) in the subject. In particular, biomarkers can include products of the activation of the complement cascade deposited on the surface of cells found in the blood, such as red blood cells, white blood cells, and platelets (e.g., cell-based complement activation products or CB-CAPS). In certain embodiments, the panel of biomarkers includes EC4d (erythrocyte-bound C4d), EC3d (erythrocyte-bound C3d), ECR1 (erythrocyte complement receptor type 1), TC3d (T-lymphocyte-bound C3d), TC4d (T-lymphocyte-bound C4d), BC4d (B-lymphocyte-bound C4d), BC3d (B-lymphocyte-bound C3d), PC4d (platelet-bound C4d), and MC4d (monocyte-bound C4d).

“Diagnostic,” as used herein, means identifying the presence or nature of a pathologic condition, such as SLE or DILE. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In one embodiment, the present invention provides a method for diagnosing SLE by determining the level of at least one biomarker in a sample from a subject. If the level of the first biomarker is within a predetermined level, then the subject is diagnosed with SLE. However, if the level of the first biomarker is outside the predetermined level, then the level of a second biomarker is determined in the sample. The subject is diagnosed with SLE if the level of the second biomarker is within a predetermined level. In some embodiments, the first and second biomarker are different and are selected from the group consisting of BC4d, EC4d, PC4d and TC4d. In one particular embodiment, the first biomarker is EC4d and the second biomarker is BC4d.

In some embodiments, the method further comprises determining the level of a third biomarker if the level of the second biomarker is outside of the second predetermined level. In such embodiments, if the level of the third biomarker is within a third predetermined level, the subject is diagnosed with SLE. Preferably, the third biomarker is different from the first and second biomarker. In certain embodiments, the third biomarker is EC4d, ECR1, or a combination thereof.

Thus, in certain embodiments, a method for diagnosing SLE comprises determining the level of BC4d in a sample from a subject, wherein if the level of BC4d is within a first predetermined level, the subject is diagnosed with SLE. If the level of BC4d is outside the predetermined level, then the level of PC4d is determined. The subject is diagnosed with SLE if the level of PC4d is within a second predetermined level. If the level of PC4d is outside the second pre-determined level, then the level of EC4d, ECR1, or a combination thereof is determined. If the level of EC4d, ECR1, or a combination thereof is within a third predetermined level, then the subject is diagnosed with SLE.

In another embodiment, a method for diagnosing SLE comprises determining the level of TC4d in sample from a subject, wherein if the level of TC4d is within a first predetermined level, the subject is diagnosed with SLE. If the level of TC4d is outside the predetermined level, then the level of PC4d is determined. The subject is diagnosed with SLE if the level of PC4d is within a second predetermined level. If the level of PC4d is outside the second pre-determined level, then the level of EC4d, ECR1, or a combination thereof is determined. If the level of EC4d, ECR1, or a combination thereof is within a third predetermined level, then the subject is diagnosed with SLE.

In yet another embodiment, a method for diagnosing SLE comprises determining the level of PC4d in sample from a subject, wherein if the level of PC4d is within a first predetermined level, the subject is diagnosed with SLE. If the level of PC4d is outside the predetermined level, then the level of TC4d is determined. The subject is diagnosed with SLE if the level of TC4d is within a second predetermined level. If the level of TC4d is outside the second pre-determined level, then the level of EC4d, ECR1, or a combination thereof is determined. If the level of EC4d, ECR1, or a combination thereof is within a third predetermined level, then the subject is diagnosed with SLE.

In certain embodiments, the method comprises determining the level of additional biomarkers (e.g., third and fourth biomarkers) if the level of the second biomarker is outside a pre-determined level or range. For instance, a method of diagnosing SLE comprises determining the level of BC4d in a sample from a subject. If the level of BC4d is within a predetermined level, then the subject is diagnosed with SLE. However, if the level of BC4d is outside the predetermined level, then the level of PC4d is determined in the sample. If the level of PC4d is outside a predetermined level or range, then the level of a third biomarker (e.g., TC4d) is determined. If the level of the third biomarker falls outside a predetermined level or range, then a fourth biomarker is determined (e.g., EC4d, ECR1, or both). Thus, in certain embodiments in which the determined level of the first and second biomarkers do not provide a definitive diagnosis for SLE, the levels of additional biomarkers can be determined.

In another embodiment, the present invention provides a method for diagnosing SLE comprising determining the level of EC4d and EC3d in a sample from a subject. The subject is diagnosed as having SLE based on the levels of EC4d and EC3d. In some embodiments, the method further comprises determining the level of ECR1 in the sample from the subject. The diagnosis of SLE is based on all three levels of EC4d, EC3d, and ECR1. In other embodiments, the method further comprises determining the level of at least one other biomarker in the sample in addition to EC4d and EC3d. The additional biomarker can be PC4d, TC3d, TC4d, BC3d, and MC4d. The subject is diagnosed with SLE based on the levels of EC4d, EC3d, and the at least one additional biomarker.

The present invention also provides a method for facilitating diagnosis of SLE. In one embodiment, the method comprises determining the level of a first biomarker in a sample from a subject, wherein if the level of the first biomarker is outside a predetermined level, then the level of a second biomarker is determined, and providing the level of the first and second biomarker to an entity for diagnosis of SLE. Preferably, the first and second biomarker are different and can be selected from the group consisting of BC4d, EC4d, PC4d and TC4d. In one embodiment, the first biomarker is EC4d and the second biomarker is BC4d.

In another embodiment, the method further comprises determining the level of a third biomarker if the level of the second biomarker is outside of the second predetermined level. The third biomarker will generally be different from the first and second biomarker. In some embodiments, the third biomarker is EC4d, ECR1, or a combination thereof. The levels of all three biomarkers are provided to an entity to facilitate the diagnosis of SLE. The entity can be, but is not limited to, a clinical laboratory, a hospital, a clinician (e.g., a physician, a physician's assistant, a nurse practitioner), and an urgent care clinic.

In some embodiments, a method for facilitating diagnosis of SLE comprises determining the level of BC4d in sample from a subject, wherein if the level of BC4d is outside a first predetermined level, then the level of PC4d is determined. If the level of PC4d is outside the second pre-determined level, then the level of EC4d, ECR1, or a combination thereof is determined. The levels of all three biomarkers are provided to an entity for diagnosis of SLE.

In another embodiment, the method for facilitating diagnosis of SLE comprises determining the level of TC4d in sample from a subject, wherein if the level of TC4d is outside a first predetermined level, then the level of PC4d is determined. If the level of PC4d is outside the second pre-determined level, then the level of EC4d, ECR1, or a combination thereof is determined. The levels of all three biomarkers are subsequently provided to an entity for diagnosis of SLE.

In still another embodiment, the method for facilitating diagnosis of SLE comprises determining the level of PC4d in sample from a subject, wherein if the level of PC4d is outside a first predetermined level, then the level of TC4d is determined. If the level of TC4d is outside the second pre-determined level, then the level of EC4d, ECR1, or a combination thereof is determined. The levels of all three biomarkers are then provided to an entity for diagnosis of SLE.

In some embodiments, the method for facilitating diagnosis of SLE comprises determining the level of additional biomarkers (e.g., third and fourth biomarkers) if the level of the second biomarker falls outside a predetermined level or range. For example, a method for facilitating diagnosis of SLE comprises determining the level of BC4d in a sample from a subject. If the level of BC4d is outside a predetermined level, then the level of PC4d is determined in the sample. If the level of PC4d is outside a predetermined level or range, then the level of a third biomarker (e.g., TC4d) is determined. If the level of the third biomarker falls outside a predetermined level or range, then a fourth biomarker is determined (e.g., EC4d, ECR1, or both). Thus, in certain embodiments in which the determined level of the first and second biomarkers do not provide a definitive diagnosis for SLE, the levels of additional biomarkers can be determined and provided to the entity for diagnosis.

Any of the diagnostic methods described herein can be used in combination with traditional diagnostic assays for SLE, such as serum C3 or C4 levels or presence of anti-dsDNA or anti-nuclear antibodies. As discussed previously, the diagnostic methods of the invention enhance the specificity and sensitivity of these conventional assays. The methods described herein are particularly useful when the conventional methods provide inconclusive results in diagnosing SLE. For instance, in one embodiment, the diagnostic methods of the invention are performed in a subject that is negative for the antinuclear antibody test or anti-double stranded DNA test.

The present invention also includes a method of detecting the development of drug-induced lupus erythematosus (DILE) in a subject who is being treated with a pharmacological agent. In one embodiment, the method comprises determining the level of at least one biomarker in a sample from the subject and determining whether the subject has DILE based on the level of the at least one biomarker. The biomarker can be selected from the group consisting of BC4d, EC4d, PC4d, TC4d, ECR1, MC4d, TC3d, BC3d, and combinations thereof. In some embodiments, multiple biomarkers can determined in the sample to determine the presence of DILE in a subject. For instance, two or more, three or more, four or more, or five or more biomarkers can be determined in the sample from the subject. In certain embodiments, a signature pattern of biomarkers is determined in the sample to assess whether DILE has developed in the subject. The diagnostic method can be performed at various time points during the course of the subject's drug treatment to assess the development or remission of DILE.

The diagnostic method for DILE can be performed in any subject being treated with any pharmacological agent. In preferred embodiments, the subject is being treated with a pharmacological agent known to be associated with DILE. Such agents include, but are not limited to, acebutolol, amiodarone, bupropion, captopril, carbamazepine, chlorpromazine, diltiazem, docetaxel. ethosuximide, gemfibrozil, glyburide, gold salt, griseofulvin, hydantoins, hydralazine, hydroxychloroquine, isoniazid, leuprolide acetate, lithium, lovastatin, mephenytoin, methyldopa, minocycline, nitrofurantoin, olanzapine, ophthalmic timolol, oral contraceptives, penicillamine, phenytoin, practolol, procainamide, propylthiouracil, quinidine, reserpine, rifampin, Simvastatin, sulfasalazine, tetracycline, ticlopidin, tiotropium bromide inhaler, trimethadione, valproate, and voriconazole. In one embodiment, the subject is treated with a biologic (e.g., genetically-engineered protein) for disorders of the immune system. For instance, the biologic can include Humira, Remicade, Enbrel, Kineret, Rituxan, or other biologics that affect the activity or expression of a cytokine.

The diagnostic methods described herein employ comparisons between a measured level of a biomarker and a pre-determined level or range. As used herein, a “pre-determined level” or “pre-determined range” refers to a value or range of values that can be determined from the quantity or amount (e.g., absolute value or concentration) of a particular biomarker measured in a population of control subjects (i.e. healthy subjects) or a population of subjects afflicted with an autoimmune disease other than SLE. A pre-determined level or pre-determined range can be selected by calculating the value or range of values that achieves the greatest statistical significance for a given set of amounts or quantities for a particular biomarker. See Example 1 for a particular example of such a method. A pre-determined level or pre-determined range can also be determined by calculating a level or range of biomarker quantities for which greater than 50%, 60%, 70%, 75%, 80%, 85%, 90%, or 95% of patients having a quantity of biomarker within that level or range have SLE. By way of example, a BC4d quantity of greater than 25.09, as determined by a recursive partition approach, was indicative of 95% of patients having SLE (see Example 1). Thus, in this particular example, a pre-determined level for the BC4d biomarker could be set at 25.09 as measured by FACs analysis. Samples in which the level of biomarker does not fall within the pre-determined range or pre-determined level, may require the measurement of an additional biomarker before a diagnosis of SLE can be made.

Particularly suitable samples for use in the methods of the invention are blood samples. Blood samples are preferably treated with EDTA (ethylenediaminetetraacetate) to inhibit complement activation. Samples can be maintained at room temperature or stored at 4° C. In some embodiments, the whole blood sample may be fractionated into different components. For instance, in one embodiment, red blood cells are separated from other cell types in the sample by differential centrifugation. Analysis of complement activation products bound to erythrocytes (e.g., EC3d, EC4d, and ECR1) can be performed on the isolated red blood cells. In another embodiment, the platelet fraction is separated from other blood components to allow analysis of platelet-bound complement activation products, such as PC4d. Platelet isolation can be performed with methods known in the art, including differential centrifugation or immunoprecipitation using antibodies specific for platelets (e.g., CD42b).

In some embodiments, the white blood cells are isolated from other components of the blood sample. For example, white blood cells (the buffy coat) can be isolated from plasma and from red blood cells by centrifugation. Each type of white blood cell (e.g. lymphocyte, monocyte, etc.) can be isolated through the use of antibodies against known cell surface markers that are specific for that cell type. Antibodies against cell surface markers of white blood cells are known to those of skill in the art. For instance, monoclonal antibodies specific for cell surface markers CD3, CD4, CD8, and CD19 are commercially available and can be used to select lymphocytes. Analysis for complement activation products found on the surface of white blood cells, such as BC4d, BC3d, TC4d, TC3d, and MC4d, can be performed in an isolated fraction of white blood cells.

The level (e.g., quantity or amount) of a particular biomarker can be measured in the sample using a variety of methods known to those of skill in the art. Such methods include, but are not limited to, flow cytometry, ELISA using red blood cell, platelet, or white blood cell lysates (e.g., lymphocyte or monocyte lysates), and radioimmunoassay. In one embodiment, the determination of the level of C4d, C3d, and CR1 is made using flow cytometric methods, with measurements taken by direct or indirect immunofluorescence using polyclonal or monoclonal antibodies specific for each of the molecules. Each of these molecules can be measured with a separate sample (e.g., red blood cell-, white blood cell-, or platelet-specific fractions) or using a single sample (e.g., whole blood).

The present invention also provides kits and combinations of tests for diagnosing SLE and/or DILE. In one embodiment, the present invention includes a combination of tests useful for diagnosing SLE comprising a first test for the level of EC4d, a second test for the level of EC3d, and a third test for the level of ECR1. In some embodiments, the combination further comprises at least one additional test for another biomarker, such as PC4d, TC3d, TC4d, BC3d, and MC4d. The kits or tests for determining the level of particular biomarkers include the various reagents for performing the measurements according to the methods described herein. For instance, in one embodiment, the kits or tests include reagents for performing immunofluorescence assays for each of the biomarkers, such as a conjugate of a monoclonal antibody specific for complement component C3d with a fluorescent moiety, a conjugate of a monoclonal antibody specific for complement component C4d with a different fluorescent moiety, and a conjugate of a monoclonal antibody specific for complement receptor CR1 with a third fluorescent moiety. Additionally, the kits can comprise such other material as may be needed in carrying out assays of this type, for example, buffers, radiolabelled antibodies, colorimeter reagents, instructions for separating different cell fractions from whole blood, and instructions for diagnosing SLE based on particular pre-determined levels of the biomarkers.

In another embodiment, the kits or tests include reagents for performing other standard assays for each of the biomarkers, such as ELISA or radioimmunoassays. In such embodiments, the kits or tests comprise monoclonal antibodies specific for C3d, C4d, and CR1 conjugated with appropriate labels such as radioactive iodine, avidin, biotin or enzymes such as peroxidase. The kits can additionally comprise buffers, substrates for antibody-conjugated enzymes, instructions for separating different cell fractions from whole blood, and instructions for diagnosing SLE based on particular pre-determined levels of the biomarkers.

The methods of the invention as described herein can be carried out manually or may be used in conjunction with an automated system or computer. For instance, the methods can be performed using an automated system, in which a subject's blood sample is analyzed to make the necessary determination or determinations of levels of particular biomarkers, and the comparison with the pre-determined level or pre-determined range is carried out automatically by software appropriate for that purpose. Computer software, or computer-readable media for use in the methods of this invention include: a computer readable medium comprising: (a) code for receiving data corresponding to a determination of complement component C4d deposited on surfaces of red blood cells, platelets, or lymphocytes (B cells and T cells); (b) code for retrieving a pre-determined level for complement component C4d deposited on surfaces of such cells of individuals; and (c) code for comparing the data in (a) with the pre-determined level of (b) to make a determination whether an accurate SLE diagnosis can be made or whether additional measurements of other biomarkers are required. In some embodiments, the computer readable medium further comprises (d) code for receiving data corresponding to a determination of complement component C3d deposited on surfaces of red blood cells, platelets, or lymphocytes (B cells and T cells); (e) code for retrieving a pre-determined level for complement component C3d deposited on surfaces of such cells of individuals; and (f) code for comparing the data in (d) with the pre-determined levels of (e). In other embodiments, the computer readable medium further comprises (g) code for receiving data corresponding to a determination of complement receptor CR1 deposited on surfaces of red blood cells; (h) code for retrieving a pre-determined level for complement receptor CR1 deposited on surfaces of red blood cells of individuals; and (i) code for comparing the data in (g) with the pre-determined level of (h).

In certain embodiments of the invention, one or more pre-determined levels or pre-determined ranges of biomarker levels may be stored in a memory associated with a digital computer. After data corresponding to a determination of complement C3d, C4d, or CR1 is obtained (e.g., from an appropriate analytical instrument), the digital computer can compare the measured biomarker data with one or more appropriate pre-determined levels or pre-determined ranges. After the comparisons take place, the digital computer can automatically calculate if the data is indicative of SLE diagnosis. Thus, the present invention also includes a collection of results in a readable format useful for diagnosing SLE comprising the level of EC4d, the level of EC3d, and the level of ECR1.

Accordingly, some embodiments of the invention may be embodied by computer code that is executed by a digital computer. The digital computer may be a micro, mini or large frame computer using any standard or specialized operating system such as a Windows based operating system. The code may be stored on any suitable computer readable media. Examples of computer readable media include magnetic, electronic, or optical disks, tapes, sticks, chips, etc. The code may also be written by those of ordinary skill in the art and in any suitable computer programming language including, C, C++, etc.

This invention is further illustrated by the following additional examples that should not be construed as limiting. The contents of all references, patents and published patent applications cited throughout this application are incorporated herein by reference in their entireties.

### EXAMPLES

### Example 1

### A Diagnostic Panel Using EC4d, ECr1, BC4d, TC4d and PC4d

A diagnostic rule was constructed from a dendrogram assessing the predictive value of a panel of CB-CAPS (e.g., EC4d, ECR1, BC4d, TC4d and PC4d) by classifying as SLE any patient in a terminal node whose percentage of SLE patients exceeds a selected cutoff. For example, a simple such rule was to classify as SLE any patient in a terminal node that is more than 50% SLE. Higher sensitivity can be achieved by reducing this 50% cutoff, and higher specificity can be achieved by increasing it.

In this Example, two perspectives were addressed: (1) Diagnosis of SLE against Other Autoimmune Diseases and (2) Diagnosis of SLE against Control (e.g., healthy subjects). In forming the dendogram, the program (e.g., recursive partitioning package FIRM) was set to allow up to a three-way split on any predictor.

### SLE vs Other Autoimmune Disease

The program selected BC4d (B-lymphocyte-bound C4d) for its initial split, creating three patient groups:
(1) Those with BC4d>25.09, comprising 168 patients, 95% of whom were SLE
(2) Those with BC4d between 5.2 and 25.09, a group of 235 patients, 68% of whom were SLE
(3) Those whose BC4d was below 5.2, or who did not have a BC4d measurement, of whom there were 491 patients, 27% of whom were SLE.

Based on the above analysis, patients in the first two groups would both be diagnosed as SLE. Thus, the distinction drawn between BC4d above or below 25.09 does not change the diagnosis, but merely the confidence in the diagnosis.

At the next level, a split was made on the third group (“BC4d≦5.2 or no BC4d measurement” group), extracting 32 patients, 72% of whom were SLE, on the basis of a positive PC4d (Platelet-bound C4d). There were 11 “Other” patients with positive PC4d in the data set; 9 of them were included in this node.

The remaining 459 patients then split by BC4d, a group of 35, 57% of whom were SLE, and then the remaining 424 patients were split using EC4d (erythrocyte-bound C4d) and ECR1 (erythrocyte CR1 receptor). The terminal nodes of the dendrogram with the initial split based on BC4d level had the following patient numbers and percent SLE as shown in Table 1:

TABLE 1
Nodes of Dendrogram for SLE vs. Other Autoimmune
with BC4d as Initial Split

| Node number | Count | % SLE |
|---|---|---|
| 3 | 235 | 68 |
| 4 | 168 | 95 |
| 6 | 32 | 72 |
| 7 | 35 | 57 |
| 9 | 311 | 15 |
| 11 | 20 | 70 |
| 12 | 22 | 68 |
| 13 | 71 | 20 |

### SLE vs Control

The initial split in this dendrogram was on EC4d (erythrocyte-bound C4d), splitting three ways:

(1) 304 patients with EC4d either missing or above 6.26, of whom 92% are SLE;

(2) 270 patients with EC4d between 3.24 and 6.26, 44% of whom are SLE;

(3) 184 patients with EC4d below 3.24, 28% of whom are SLE.

Each of these three groups was then refined by splits on BC4d. The terminal nodes of this dendrogram had the following patient numbers and percent SLE as shown in Table 2:

TABLE 2
Nodes of Dendrogram for SLE vs. Control with EC4d as Initial Split

| Node number | Count | % SLE |
|---|---|---|
| 5 | 128 | 17 |
| 6 | 56 | 54 |
| 7 | 10 | 70 |
| 8 | 144 | 24 |
| 9 | 116 | 67 |
| 10 | 118 | 79 |
| 11 | 186 | 100 |

### Alternative Possible Splits

The dendrograms are generated by selecting whichever of the predictors provides the greatest statistical significance for splitting the group currently being analyzed. It is however interesting to know how the runners-up would have performed on splitting the cases. The output for the initial split for SLE v. Other Autoimmune Disease included the following information:

TABLE 3
Alternative Predictors for Initial Split for SLE vs. Other Autoimmune

| | P values | | |
|---|---|---|---|
| | | Mult. | |
| Predictor | Bonferroni | Comparisons | Breakdown into Classes |
| ECR1 | 1.61E−10% | 9.95E−04% | <=4.715;; <=10.935;; ?, >10.935 |
| EC4d | 2.23E−25% | 3.79E−15% | <=6.156;; <=15.61;; ?, >15.61 |
| PC4d+ | 1.78E−13% | 1.78E−13% | ?,;; <=0.0;; >0.0 |
| BC4d | 2.45E−53% | 9.39E−40% | ?, <=5.2;; <=25.09;; >25.09 |
| TC4d | 1.47E−51% | 3.89E−38% | ?, <=0.87;; <=4.39;; >4.39 |

The column headed “Bonferroni” gives the statistical significance of the split that could be made on that predictor. Under “breakdown into classes” one can see where the divisions would have been made. For example, the record for BC4d (?,<=5.2;; <=25.09;; >25.09) shows the three-way grouping ≦5.2, 5.2 to 25.09, >25.09, with the cases who did not have a BC4d value (the ? group) pooled with the ≦5.2 group.

BC4d had the smallest P value, 2.45×10⁻⁵³, and so was used for the first dendrogram split. But TC4d (T-lymphocyte-bound C4d) was not far behind; its P value was 1.47×10⁻⁵¹. Had TC4d been used to split the group, it would also have been split three ways, with cut points at 0.87 and 4.39.

While the other three markers were all statistically highly significant, they all trailed these two by a wide margin.

The output for the initial split for SLE v. Control dendrogram included the following information:

TABLE 4
Alternative Predictors for Initial Split for SLE vs. Control

| | P values | | |
|---|---|---|---|
| | | Mult. | |
| Predictor | Bonferroni | Comparisons | Breakdown into Classes |
| ECR1 | 1.94E−21% | 6.38E−12% | <=6.555;; <=10.85;; ?, >10.85 |
| EC4d | 1.24E−44% | 7.01E−32% | <=3.24;; <=6.26;; ?, >6.26 |
| PC4d+ | 5.91E−13% | 2.97E−12% | ?, <=0.0;; >0.0 |
| BC4d | 1.20E−34% | 4.75E−23% | <=15.41;; ?, <=15.49;; >15.49 |
| TC4d | 5.86E−26% | 1.24E−15% | ?, <=0.81;; <=5.04;; >5.04 |

In this analysis, there was no close runner-up. The three-way split on EC4d was much more significant with a p value of 1.24×10⁻⁴⁴ than any of the other markers. BC4d was a distant second with a p value of 1.20×10⁻³⁴.

This result does not necessarily mean that EC4d is a better indicator of SLE against asymptomatic controls. The fact that many more patients had measured EC4d than BC4d or TC4d may be the root cause of its greater statistical significance because of the implied larger sample size.

### Example 2

### Use of Pc4d as an Initial Predictor of SLE

PC4d (platelet-bound C4d) has been described previously as a first splitter, to use its high specificity to isolate the 19% of SLE patients whose PC4d is positive. This would yield a dendrogram whose first split was on PC4d with the other markers being used to refine the daughter node with negative PC4d.

The software does not allow the user to force particular splits, and so cannot be used to construct such a dendrogram directly. We can instead get the performance of this dendrogram in two stages: The initial split by PC4d gives:

| PC4d positive | SLE | Other | Total |
|---|---|---|---|
| 0 | 356 | 394 | 750 |
| 1 | 85 | 11 | 96 |
| Total | 441 | 405 | 846 |

Cases Included 846; Missing Cases 48. The group with positive PC4d numbers 96, of whom 89% are SLE.

A dendrogram is then created with the remaining 798 patients, which has an initial three-way split using TC4d. The split is subsequently refined using EC4d and ECR1 levels.

The overall set of terminal nodes (including the initial split on PC4d) gives:

TABLE 5
Nodes of Dendrogram for SLE vs. Other Autoimmune
with PC4d as Initial Split

| Node number | Count | % SLE |
|---|---|---|
| Preliminary | 96 | 89 |
| 3 | 183 | 63 |
| 5 | 357 | 17 |
| 7 | 22 | 68 |
| 8 | 73 | 71 |
| 9 | 113 | 95 |
| 10 | 11 | 91 |
| 11 | 39 | 18 |

Comparison of SLE v. Other Autoimmune Dendrographic Models (BC4d v. PC4d as Initial Split)

As illustrated in Example 1, one analysis started as a dendrogram with eight terminal nodes for distinguishing SLE from other autoimmune disorders. The first split in the dendrogram was on BC4d, a biomarker not measured in all patient data. Then PC4d, EC4d and ECR1 were used, giving a dendrogram with 8 terminal nodes. This leads to the following:

TABLE 6
Diagnostic Rule based on Dendrogram in Example 1 (Split by BC4d)

| Node | Count | SLE | Other | Diagnosis |
|---|---|---|---|---|
| 3 | 235 | 159 | 76 | SLE |
| 4 | 168 | 160 | 8 | SLE |
| 6 | 32 | 23 | 9 | SLE |
| 7 | 35 | 20 | 15 | SLE |
| 9 | 311 | 46 | 265 | Other |
| 11 | 20 | 14 | 6 | SLE |
| 12 | 22 | 15 | 7 | SLE |
| 13 | 71 | 14 | 57 | Other |

The performance of this dendrogram is summarized using conventional diagnostic performance statistics (Table 7). The positive and negative predictive values (PPV and NPV, respectively) require an assumption on prevalence. The calculations below assume that the prevalence is the proportion of patients within the data set who have SLE, that is, 50.4% which corresponds to 451 out of 894 patients.

TABLE 7
Predictive Diagnostic Value of Dendrogram in Example 1 (Split by BC4d)

| | | True Status | | | | |
|---|---|---|---|---|---|---|
| | | SLE | Other | Total | PPV | NPV |
| Test | SLE | 391 | 121 | 512 | 76.4 | |
| | Not SLE | 60 | 322 | 382 | | 84.3 |
| | Total | 451 | 443 | 894 | | |
| | Sensitivity | 86.7 | | | | |
| | Specificity | | 72.7 | | | |

A second dendrographic model (illustrated in this Example) was used which capitalized on the high specificity of the PC4d marker, making an initial split using PC4d. Subsequent splits used TC4d, EC4d and ECR1, again leading to 8 terminal nodes with the following make up:

TABLE 8
Diagnostic Rule based on Dendrogram in Example 2 (Split by PC4d)

| Node | Count | SLE | Other | Diagnosis |
|---|---|---|---|---|
| Preliminary | 96 | 85 | 11 | SLE |
| 3 | 183 | 115 | 68 | SLE |
| 5 | 357 | 60 | 297 | Other |
| 7 | 22 | 15 | 7 | SLE |
| 8 | 73 | 52 | 21 | SLE |
| 9 | 113 | 107 | 6 | SLE |
| 10 | 11 | 10 | 1 | SLE |
| 11 | 39 | 7 | 32 | Other |

The diagnostic performance of this dendrogram is:

TABLE 9
Predictive Diagnostic Value of Dendrogram in Example 2 (Split by PC4d)

| | | True Status | | | | |
|---|---|---|---|---|---|---|
| | | SLE | Other | Total | PPV | NPV |
| Test | SLE | 384 | 114 | 498 | 77.1 | |
| | Not SLE | 67 | 329 | 396 | | 83.1 |
| | Total | 451 | 443 | 894 | | |
| | Sensitivity | 86.7 | | | | |
| | Specificity | | 72.7 | | | |

Accordingly, the two dendrograms have very similar performance. Both PPV and NPV on the diagnostic panel are in the 80% range for diagnosing SLE versus “other” diseases, which clinically is an important measure.

### Example 3

### Correlation of CB-CAPS Biomarkers with SLAM and SLEDAI Indices

One difficulty in diagnosing SLE is that it evolves over time. Consequently, disease activity must be assessed at each time point of interest. As previously described, lupus specialists have relied largely on various “scales” or “indices” such as the SLEDAI or SLAM. When these measures are used to evaluate a new modality and if they are “noisy” or do not actually correlate well with real disease activity, then the new modality may not look particularly useful or effective even though in reality it does reflect the biological and physiological status of the disease.

Longitudinal monitoring of SLE involves the relationship between the course of a patient's SLE clinical status and the contemporaneous record of biomarkers. In a typical longitudinal study of SLE disease activity, three scales are used as “predicates”:
1. SLM_Tot—this is the SLAM, which does NOT include the traditional markers, C3, C4 and anti-dsDNA
2. SLEDAI_Tot—this is the SLEDAI, which does include the traditional C3, C4 and anti-dsDNA markers
3. SLEDAI_New—this is a modified SLEDAI, which does NOT include the traditional markers, C3, C4 and anti-dsDNA

While SLEDAI_New might be considered a “pure” statistical comparison, it is impossible to say if it truly correlates with real disease activity. Unlike the SLAM, it is not an established scale in wide-spread use. Thus, it is believed that the best and most objective way to look at performance of CB-CAPs versus C3, C4 and anti-dsDNA is SLM_Tot.

Previous work has used an analysis of covariance (ANCOVA) model. Writing yᵢₜ for a clinical status measure such as SLEDAI measured on patient i at time t, and xᵢₜ for the contemporaneous value of a biomarker, the ANCOVA model is:

yᵢₜ=αᵢ+βxᵢₜ+eᵢₜ

where αᵢ is a patient-specific intercept, β is a slope, assumed to be the same for all patients, and eᵢₜ is a random error term.

The statistical significance of the biomarker can be assessed by a P value for the null hypothesis that β=0, and the predictive power of the biomarker assessed by the partial correlation between the biomarker and the clinical status measure, adjusted for the patient to patient differences.

In view of the highly right-skew statistical distribution of most of the CB-CAP biomarkers, all except ECR1 were log-transformed for the analysis.

Table 10 shows the results of this analysis for three clinical measures: SLAM_Tot, SLEDAI_New, and SLEDAI_Tot, and the suite of biomarkers. Also shown are the number of patients and total number of visits from which these values were calculated.

TABLE 10
Partial Correlations of Biomarkers and Clinical Status

| Marker | | | | SLAM_Tot | SLEDAI_New | SLEDAI_Tot |
|---|---|---|---|---|---|---|
| C3 | patients | 314 | partial r | −0.133 | −0.125 | −0.289 |
| | visits | 1309 | P value | 2.50E−05 | 7.70E−05 | 2.20E−13 |
| C4 | patients | 314 | partial r | −0.142 | −0.123 | −0.266 |
| | visits | 1307 | P value | 7.30E−06 | 9.70E−05 | 2.30E−13 |
| dsDNA | patients | 299 | partial r | 0.167 | 0.081 | 0.346 |
| | visits | 1121 | P value | 1.60E−06 | 0.0207 | 2.50E−13 |
| ECR1 | patients | 320 | partial r | −0.055 | −0.059 | −0.100 |
| | visits | 1316 | P value | 0.0846 | 0.0635 | 0.0015 |
| EC4d | patients | 320 | partial r | 0.207 | 0.070 | 0.132 |
| | visits | 1318 | P value | 3.90E−11 | 0.0269 | 2.80E−05 |
| PC4d+ | patients | 317 | partial r | −0.012 | 0.068 | 0.077 |
| | visits | 1310 | P value | 0.7079 | 0.0329 | 0.0154 |
| EC3d | patients | 320 | partial r | 0.199 | 0.064 | 0.151 |
| | visits | 1318 | P value | 2.40E−10 | 0.0419 | 1.70E−06 |
| PC3d | patients | 263 | partial r | −0.059 | 0.005 | 0.047 |
| | visits | 713 | P value | 0.2154 | 0.9177 | 0.3206 |
| RC4d | patients | 276 | partial r | −0.039 | 0.007 | −0.002 |
| | visits | 991 | P value | 0.3037 | 0.8623 | 0.9686 |
| RC3d | patients | 276 | partial r | 0.015 | 0.030 | 0.057 |
| | visits | 991 | P value | 0.6837 | 0.4274 | 0.1308 |
| TC4d | patients | 286 | partial r | −0.089 | −0.066 | −0.065 |
| | visits | 1105 | P value | 0.0113 | 0.0590 | 0.0625 |
| TC3d | patients | 285 | partial r | 0.019 | −0.104 | −0.049 |
| | visits | 1103 | P value | 0.5812 | 0.0029 | 0.1629 |
| TC4d_4 | patients | 284 | partial r | −0.025 | 0.014 | −0.014 |
| | visits | 1076 | P value | 0.4907 | 0.7001 | 0.7013 |
| TC3d_4 | patients | 283 | partial r | 0.001 | −0.040 | −0.020 |
| | visits | 1074 | P value | 0.9862 | 0.2608 | 0.5718 |
| TC4d_8 | patients | 284 | partial r | −0.113 | −0.057 | −0.065 |
| | visits | 1062 | P value | 0.0016 | 0.1141 | 0.0683 |
| TC3d_8 | patients | 284 | partial r | −0.049 | −0.029 | −0.014 |
| | visits | 1060 | P value | 0.1692 | 0.4202 | 0.7025 |
| BC4d | patients | 285 | partial r | −0.031 | −0.018 | −0.025 |
| | visits | 1085 | P value | 0.3756 | 0.6157 | 0.4764 |
| BC3d | patients | 285 | partial r | −0.050 | −0.081 | −0.100 |
| | visits | 1084 | P value | 0.1617 | 0.0224 | 0.0048 |
| MC4d | patients | 286 | partial r | 0.070 | 0.046 | 0.055 |
| | visits | 1105 | P value | 0.0464 | 0.1893 | 0.1164 |
| MC3d | patients | 286 | partial r | 0.064 | 0.022 | 0.034 |
| | visits | 1105 | P value | 0.0683 | 0.5316 | 0.3367 |
| GC4d | patients | 285 | partial r | 0.032 | −0.059 | −0.019 |
| | visits | 1101 | P value | 0.3624 | 0.0927 | 0.5882 |
| GC3d | patients | 285 | partial r | 0.069 | −0.008 | 0.051 |
| | visits | 1102 | P value | 0.0472 | 0.8287 | 0.1435 |

As evident from the results shown in Table 10, all three measures are highly statistically correlated with serum C3, C4 and anti-dsDNA. In addition, striking significances among the CB-CAP biomarkers are those involving EC4d and EC3d on SLM_Tot and SLEDAI_Tot and less strongly SLEDAI_New, and ECR1 on SLEDAI_Tot. Other 5% significant but weaker correlations are seen with PC4d, TC3d, TC4d_{—}8, BC3d and MC4d (Monocyte-bound C4d) and one or two of the clinical measures.

### Variable Correlation

In considering panels for SLE monitoring, the correlation of different measures were assessed. Table 11 shows the within-patient correlations among the three disease markers, and of each of them with the key biomarkers. Where the same pair is shown, these correlations should be the same as those in Table 10, however when they are not identical, the differences may be due to missing information. The values in Table 11 are based on 1033 records on 288 patients in which SLM_Tot, SLEDAI_New, SLEDAI_Tot, C3, C4, dsDNA, ECR1, EC4d, and EC3d were all measured.

TABLE 11
Correlations with Clinical Measures

| | SLM_Tot | SLEDAI_New | SLEDAI_Tot |
|---|---|---|---|
| SLM_Tot | 1.000 | 0.342 | 0.358 |
| SLEDAI_New | 0.342 | 1.000 | 0.883 |
| SLEDAI_Tot | 0.358 | 0.883 | 1.000 |
| C3 | −0.105 | −0.095 | −0.250 |
| C4 | −0.136 | −0.096 | −0.244 |
| dsDNA | 0.150 | 0.051 | 0.319 |
| ECR1 | −0.073 | −0.077 | −0.111 |
| EC4d | 0.215 | 0.056 | 0.111 |
| EC3d | 0.188 | 0.046 | 0.117 |

Table 11 confirms that SLM_Tot is relatively weakly correlated with the two SLEDAI measures, a consequence of their measuring different dimensions of the progression of SLE. SLEDAI_New shows weaker correlations with the biomarkers than does SLEDAI_Tot. This result partly reflects the correlation induced in SLEDAI_Tot by the fact that it is partly determined by C3 and C4.

The within-patient correlation between the biomarkers is shown in Table 12. There are two large correlations in this table—that between C3 and C4, and that between EC4d and EC3d. There are also a number of highly statistically significant, though much smaller, correlations.

TABLE 12
Correlation among Biomarkers

| | C3 | C4 | dsDNA | ECR1 | EC4d | EC3d |
|---|---|---|---|---|---|---|
| C3 | 1.000 | 0.603 | −0.089 | 0.153 | −0.023 | −0.083 |
| C4 | 0.603 | 1.000 | −0.132 | 0.084 | −0.173 | −0.196 |
| dsDNA | −0.089 | −0.132 | 1.000 | −0.015 | 0.112 | 0.103 |
| ECR1 | 0.153 | 0.084 | −0.015 | 1.000 | 0.239 | 0.093 |
| EC4d | −0.023 | −0.173 | 0.112 | 0.239 | 1.000 | 0.672 |
| EC3d | −0.083 | −0.196 | 0.103 | 0.093 | 0.672 | 1.000 |

### Testing for Additional Information

These correlations among the biomarkers suggest that, for the most part, they carry different information about the severity of SLE symptoms. This leads to the task of formally testing whether the CB-CAP biomarkers convey information additional to that contained in serum C3, C4 and anti-dsDNA. This can be tested by extending the analysis of covariance model to include C3, C4, anti-dsDNA, and each of the CB-CAP biomarkers:

yᵢₜ=αᵢ+γC3+δC4+ØdsDNA+βxᵢₜ+eᵢₜ

where x is one of the CB-CAP markers.

Formal testing of the hypothesis that β=0 tests the significance of the additional information, and the partial correlation and P values of the CB-CAP biomarker, corrected for subject differences and C3, C4 and dsDNA measures the additional information.

These numbers are provided in Table 13, below. These patient and visit counts are different from those previously described because of missing information—to be used in the analysis of Table 13, a visit requires values for C3, C4, anti-ds DNA as well as for the CB-CAP biomarker.

Among these relationships corrected for C3, C4 and anti-dsDNA, Table 13 shows that EC4d and EC3d retain their high statistical significance on SLM_Tot, and ECR1 is significant at the 5% level on SLEDAI_Tot. Also, T-cell markers TC4d and TC4d_{—}8, and TC3d_{—}8 are significant for SLM_Tot, and TC3d correlates significantly with SLEDAI_Tot.

Given the mostly quite low correlations among the biomarkers, it was unexpected that some of the partial correlations after correction of C3, C4 and anti-dsDNA were appreciably lower than the values in Table 10. This is an indication that the inter-correlations between biomarkers do correspond to some degree of information overlap.

TABLE 13
Additional Information in CB-CAP Biomarkers

| Marker | | | | SLAM_Tot | SLEDAI_New | SLEDAI_Tot |
|---|---|---|---|---|---|---|
| ECR1 | patients | 288 | partial r | −0.059 | −0.064 | −0.081 |
| | visits | 1033 | P value | 0.1088 | 0.0822 | 0.0272 |
| EC4d | patients | 288 | partial r | 0.191 | 0.042 | 0.063 |
| | visits | 1033 | P value | 0.0000 | 0.2578 | 0.0874 |
| EC3d | patients | 288 | partial r | 0.159 | 0.027 | 0.059 |
| | visits | 1033 | P value | 0.0000 | 0.4583 | 0.1108 |
| PC4d+ | patients | 285 | partial r | −0.036 | 0.048 | 0.063 |
| | visits | 1025 | P value | 0.3342 | 0.1917 | 0.0894 |
| PC3d | patients | 245 | partial r | −0.050 | 0.026 | 0.056 |
| | visits | 625 | P value | 0.3349 | 0.6095 | 0.2772 |
| RC4d | patients | 244 | partial r | −0.056 | −0.009 | −0.025 |
| | visits | 767 | P value | 0.2035 | 0.8377 | 0.5697 |
| RC3d | patients | 244 | partial r | 0.052 | 0.027 | 0.039 |
| | visits | 767 | P value | 0.2324 | 0.5468 | 0.3764 |
| TC4d | patients | 255 | partial r | −0.112 | −0.090 | −0.080 |
| | visits | 843 | P value | 0.0067 | 0.0298 | 0.0531 |
| TC3d | patients | 254 | partial r | −0.032 | −0.153 | −0.120 |
| | visits | 843 | P value | 0.4341 | 0.0002 | 0.0037 |
| TC4d_4 | patients | 253 | partial r | −0.038 | −0.023 | −0.033 |
| | visits | 816 | P value | 0.3719 | 0.5810 | 0.4311 |
| TC3d_4 | patients | 252 | partial r | −0.050 | −0.079 | −0.079 |
| | visits | 816 | P value | 0.2385 | 0.0620 | 0.0608 |
| TC4d_8 | patients | 253 | partial r | −0.146 | −0.066 | −0.071 |
| | visits | 803 | P value | 0.0006 | 0.1240 | 0.0955 |
| TC3d_8 | patients | 253 | partial r | −0.113 | −0.058 | −0.041 |
| | visits | 802 | P value | 0.0080 | 0.1755 | 0.3394 |
| BC4d | patients | 255 | partial r | 0.001 | −0.026 | −0.009 |
| | visits | 833 | P value | 0.9820 | 0.5397 | 0.8293 |
| BC3d | patients | 255 | partial r | −0.035 | −0.088 | −0.105 |
| | visits | 832 | P value | 0.4014 | 0.0344 | 0.0122 |
| MC4d | patients | 255 | partial r | 0.076 | −0.025 | −0.026 |
| | visits | 843 | P value | 0.0674 | 0.5408 | 0.5344 |
| MC3d | patients | 255 | partial r | 0.069 | 0.010 | −0.007 |
| | visits | 843 | P value | 0.0933 | 0.8027 | 0.8610 |
| GC4d | patients | 254 | partial r | 0.043 | −0.075 | −0.044 |
| | visits | 839 | P value | 0.3024 | 0.0723 | 0.2924 |
| GC3d | patients | 254 | partial r | 0.060 | −0.016 | 0.008 |
| | visits | 840 | P value | 0.1446 | 0.6965 | 0.8422 |

### Analysis of ECR1, EC4d or EC3d to C3, C4 and Anti-dsDNA

Table 13 demonstrates that EC4d and EC3d each add highly significant information to that in serum C3, C4 and anti-dsDNA, and that ECR1 has a more modest statistical significance. This leads to the converse question of how much additional information C3, C4 and anti-dsDNA convey in a panel of these three traditional markers plus ECR1, EC4d or EC3d.

This question can be addressed by looking at the coefficients in the analysis of covariance, their standard errors, and their t values. The following Tables 14, 15 and 16 for SLM_Tot, SLEDAI_New and SLEDAI_Tot demonstrate this analysis, respectively.

TABLE 14
Tests for Additional Information: SLM_Tot
SLM_Tot

| Pred | C3 | C4 | dsDNA | |
|---|---|---|---|---|
| | | | | ECR1 |
| coeff | −0.3674 | −0.7046 | 0.9411 | −0.0725 |
| se | 0.6706 | 0.3247 | 0.254 | 0.0452 |
| t | −0.5479 | −2.1698 | 3.7046 | −1.6057 |
| P | 0.5839 | 0.0303 | 0.0002 | 0.1088 |
| | | | | EC4d |
| coeff | −0.8714 | −0.3705 | 0.8183 | 1.686 |
| se | 0.6576 | 0.3253 | 0.2509 | 0.3186 |
| t | −1.3251 | −1.1391 | 3.2615 | 5.2916 |
| P | 0.1856 | 0.2550 | 0.0012 | 0.0000 |
| | | | | EC3d |
| coeff | −0.6402 | −0.4477 | 0.8529 | 1.7049 |
| se | 0.6584 | 0.3262 | 0.2520 | 0.3885 |
| t | −0.9724 | −1.3726 | 3.3842 | 4.3889 |
| P | 0.3312 | 0.1703 | 0.0008 | 0.0000 |

Table 14 illustrates the high statistical significance of EC4d, and that of EC3d, when these are used to augment C3, C4 and anti-dsDNA in monitoring SLM_Tot. It is striking that C3 is not statistically significant in any of these regressions, and C4 is also rather weak. The most likely explanation for this is that C3 and C4 contain strongly overlapping information, and so while it is not necessary to use both of them, one of them may be helpful.

TABLE 15
Tests for Additional Information: SLEDAI New
SLEDAI_New

| Pred | C3 | C4 | dsDNA | |
|---|---|---|---|---|
| | | | | ECR1 |
| coeff | −0.5653 | −0.3252 | 0.2179 | −0.0640 |
| se | 0.5457 | 0.2642 | 0.2067 | 0.0368 |
| t | −1.0358 | −1.2307 | 1.0539 | −1.7404 |
| P | 0.3006 | 0.2188 | 0.2923 | 0.0822 |
| | | | | EC4d |
| coeff | −0.7523 | −0.2621 | 0.1966 | 0.2989 |
| se | 0.5448 | 0.2695 | 0.2079 | 0.2640 |
| t | −1.3807 | −0.9727 | 0.9460 | 1.1324 |
| P | 0.1678 | 0.3310 | 0.3445 | 0.2578 |
| | | | | EC3d |
| coeff | −0.7062 | −0.2854 | 0.2062 | 0.2376 |
| se | 0.5427 | 0.2688 | 0.2077 | 0.3202 |
| t | −1.3013 | −1.0615 | 0.9924 | 0.7421 |
| P | 0.1936 | 0.2888 | 0.3213 | 0.4583 |

In Table 15, it is shown that none of the predictors is statistically significant. These results do not necessarily mean that the predictions are without value, but rather that the biomarkers contain enough information overlap to hide what associations there might be. This is addressed further in Example 4.

TABLE 16
Tests for Additional Information: SLEDAI_Tot
SLEDAI_Tot

| Pred | C3 | C4 | dsDNA | |
|---|---|---|---|---|
| | | | | ECR1 |
| coeff | −2.0453 | −0.7594 | 1.9312 | −0.0887 |
| se | 0.5953 | 0.2883 | 0.2255 | 0.0401 |
| t | −3.4357 | −2.6345 | 8.5642 | −2.2123 |
| P | 0.0006 | 0.0086 | 0.0000 | 0.0272 |
| | | | | EC4d |
| coeff | −2.3216 | −0.6567 | 1.8961 | 0.4929 |
| se | 0.5944 | 0.2940 | 0.2268 | 0.2880 |
| t | −3.9056 | −2.2334 | 8.3607 | 1.7115 |
| P | 0.0001 | 0.0258 | 0.0000 | 0.0874 |
| | | | | EC3d |
| coeff | −2.2587 | −0.6705 | 1.9031 | 0.5576 |
| se | 0.5919 | 0.2932 | 0.2266 | 0.3492 |
| t | −3.8158 | −2.2866 | 8.3990 | 1.5966 |
| P | 0.0001 | 0.0225 | 0.0000 | 0.1108 |

As shown in Table 16, C3, C4 and anti-dsDNA are all statistically significant. ECR1 is 5% significant, but the other two CB-CAP markers are not, on their own, significant.

### Example 4

### Diagnostic Panels Incorporating CB-CAPS Biomarkers

ECR1, EC4d and EC3d each demonstrate highly significant information in some settings. This raises the question of using these three biomarkers in a panel, along with one or more of the traditional biomarkers C3, C4 and anti-dsDNA. This possibility is explored in the following analysis of covariance fits, each using these three CB-CAP markers and a subset of C3, C4 and anti-dsDNA. Particular interest centers on the P values of C3, C4 and anti-dsDNA in these fits, as they indicate the value of adding each of these biomarkers to the CB-CAP panel.

SLM_Tot: Table 17 deals with SLM_Tot, summarizing all possible analysis of covariance models incorporating ECR1, EC4d, EC3d and any subset of C3, C4 and anti-dsDNA. The overall picture is quite straightforward: anti-dsDNA is highly significant in each of the models in which it appears, but in models in which it is included, neither C3 nor C4 is statistically significant. This result indicates that the prognostic information in C3 and C4 can be obtained from anti-dsDNA and the three CB-CAP biomarkers, and that C3 and C4 are unnecessary.

SLEDAI_New: Table 18 provides the parallel information for SLEDAI_New. In these fits, statistical significances are considerably sparser than with SLM_Tot. C3 or C4, but not both, add statistically significant information to that contained in the CB-CAP biomarkers, but anti-dsDNA does not. The baseline fit using ECR1, EC4d and EC3d is statistically significantly predictive: P=0.014, with most of the significance being due to ECR1. So there is a statistically relationship between SLEDAI_New and the panel, and this is enhanced somewhat by adding C4 (or less effectively, C3).

SLEDAI_Tot: Table 19 contains the results for SLEDAI_Tot. This table is in a sense the opposite extreme from Table 18; C3, C4 and anti-dsDNA are generally statistically significant in every model that includes them. This is partly an artifact, as the calculation of SLEDAI_Tot includes serum C3 and C4.

In these model fits, the P values of the CB-CAP biomarkers are secondary. Lack of significance on EC4d or EC3d is possibly believed to be an indication that, as the two are moderately correlated, there is a degree of substitutability between them. Small P values, however, do demonstrate the value of the marker, and small values are indeed seen in a number of the fits.

TABLE 17
CB-CAP Biomarkers Plus Traditional Markers for SLM-Tot

| SLAM_Tot | R\hat{ }2 | C3 | C4 | dsDNA |
|---|---|---|---|---|
| coeff | 0.037 | −0.506 | −0.699 | 0.942 |
| se | | 0.666 | 0.325 | 0.254 |
| t | | −0.76 | −2.151 | 3.704 |
| P | | 0.4475 | 0.0318 | 0.0002 |

| SLAM_Tot | R\hat{ }2 | ECR1 | EC4d | EC3d |
|---|---|---|---|---|
| coeff | 0.065 | −0.157 | 1.781 | 0.680 |
| se | | 0.046 | 0.434 | 0.513 |
| t | | −3.455 | 4.107 | 1.324 |
| P | | 0.0006 | 4.50E−05 | 0.1859 |

| SLAM_Tot | R\hat{ }2 | ECR1 | EC4d | EC3d | dsDNA |
|---|---|---|---|---|---|
| coeff | 0.079 | −0.151 | 1.685 | 0.620 | 0.848 |
| se | | 0.045 | 0.432 | 0.510 | 0.249 |
| t | | −3.343 | 3.904 | 1.216 | 3.413 |
| P | | 0.0009 | 0.0001 | 0.2244 | 0.0007 |
| SLAM_Tot | R\hat{ }2 | ECR1 | EC4d | EC3d | C4 |
| coeff | 0.071 | −0.145 | 1.697 | 0.566 | −0.590 |
| se | | 0.046 | 0.434 | 0.514 | 0.261 |
| t | | −3.162 | 3.91 | 1.101 | −2.26 |
| P | | 0.0016 | 0.0001 | 0.2711 | 0.0241 |

| SLAM_Tot | R\hat{ }2 | ECR1 | EC4d | EC3d | C4 | dsDNA |
|---|---|---|---|---|---|---|
| coeff | 0.084 | −0.141 | 1.619 | 0.528 | −0.500 | 0.797 |
| se | | 0.045 | 0.432 | 0.511 | 0.261 | 0.250 |
| t | | −3.097 | 3.747 | 1.032 | −1.918 | 3.194 |
| P | | 0.0020 | 0.0002 | 0.3024 | 0.0554 | 0.0015 |

| SLAM_Tot | R\hat{ }2 | ECR1 | EC4d | EC3d | C3 |
|---|---|---|---|---|---|
| coeff | 0.071 | −0.142 | 1.786 | 0.594 | −1.155 |
| se | | 0.046 | 0.433 | 0.514 | 0.531 |
| t | | −3.081 | 4.128 | 1.157 | −2.177 |
| P | | 0.0021 | 4.10E−05 | 0.2475 | 0.0298 |

| SLAM_Tot | R\hat{ }2 | ECR1 | EC4d | EC3d | C3 | dsDNA |
|---|---|---|---|---|---|---|
| coeff | 0.084 | −0.138 | 1.693 | 0.548 | −1.019 | 0.811 |
| se | | 0.046 | 0.431 | 0.510 | 0.529 | 0.249 |
| t | | −3.013 | 3.931 | 1.073 | −1.928 | 3.257 |
| P | | 0.0027 | 0.0001 | 0.2838 | 0.0543 | 0.0012 |
| SLAM_Tot | R\hat{ }2 | ECR1 | EC4d | EC3d | C3 | C4 |
| coeff | 0.073 | −0.140 | 1.729 | 0.555 | −0.683 | −0.389 |
| se | | 0.046 | 0.435 | 0.515 | 0.662 | 0.325 |
| t | | −3.037 | 3.973 | 1.078 | −1.032 | −1.196 |
| P | | 0.0025 | 0.0001 | 0.2814 | 0.3025 | 0.2322 |

| SLAM_Tot | R\hat{ }2 | ECR1 | EC4d | EC3d | C3 | C4 | dsDNA |
|---|---|---|---|---|---|---|---|
| coeff | 0.085 | −0.136 | 1.649 | 0.517 | −0.644 | −0.312 | 0.793 |
| se | | 0.046 | 0.433 | 0.512 | 0.658 | 0.324 | 0.250 |
| t | | −2.979 | 3.807 | 1.01 | −0.979 | −0.961 | 3.175 |
| P | | 0.0030 | 0.0002 | 0.3128 | 0.3279 | 0.3369 | 0.0016 |

TABLE 18
CB-CAP Biomarkers Plus Traditional Markers for SLEDAI_New

| SLEDAI_New | R\hat{ }2 | C3 | C4 | dsDNA |
|---|---|---|---|---|
| coeff | 0.013 | −0.687 | −0.320 | 0.219 |
| se | | 0.542 | 0.265 | 0.207 |
| t | | −1.269 | −1.211 | 1.056 |
| P | | 0.2050 | 0.2263 | 0.2914 |
| SLEDAI_New | R\hat{ }2 | ECR1 | EC4d | EC3d |
| coeff | 0.012 | −0.095 | 0.533 | 0.036 |
| se | | 0.038 | 0.358 | 0.424 |
| t | | −2.522 | 1.488 | 0.084 |
| P | | 0.0119 | 0.1372 | 0.9328 |

| SLEDAI_New | R\hat{ }2 | ECR1 | EC4d | EC3d | dsDNA |
|---|---|---|---|---|---|
| coeff | 0.013 | −0.093 | 0.507 | 0.019 | 0.235 |
| se | | 0.038 | 0.359 | 0.424 | 0.207 |
| t | | −2.476 | 1.411 | 0.046 | 1.134 |
| P | | 0.0135 | 0.1588 | 0.9637 | 0.2570 |
| SLEDAI_New | R\hat{ }2 | ECR1 | EC4d | EC3d | C4 |
| coeff | 0.017 | −0.085 | 0.469 | −0.051 | −0.450 |
| se | | 0.038 | 0.359 | 0.425 | 0.216 |
| t | | −2.253 | 1.307 | −0.119 | −2.087 |
| P | | 0.0245 | 0.1916 | 0.9052 | 0.0372 |

| SLEDAI_New | R\hat{ }2 | ECR1 | EC4d | EC3d | C4 | dsDNA |
|---|---|---|---|---|---|---|
| coeff | 0.019 | −0.084 | 0.451 | −0.060 | −0.429 | 0.191 |
| se | | 0.038 | 0.360 | 0.426 | 0.217 | 0.208 |
| t | | −2.228 | 1.253 | −0.141 | −1.976 | 0.918 |
| P | | 0.0262 | 0.2106 | 0.8880 | 0.0485 | 0.3589 |

| SLEDAI_New | R\hat{ }2 | ECR1 | EC4d | EC3d | C3 |
|---|---|---|---|---|---|
| coeff | 0.018 | −0.082 | 0.537 | −0.035 | −0.960 |
| se | | 0.038 | 0.358 | 0.424 | 0.439 |
| t | | −2.155 | 1.503 | −0.083 | −2.19 |
| P | | 0.0315 | 0.1333 | 0.9340 | 0.0289 |

| SLEDAI_New | R\hat{ }2 | ECR1 | EC4d | EC3d | C3 | dsDNA |
|---|---|---|---|---|---|---|
| coeff | 0.019 | −0.081 | 0.514 | −0.047 | −0.927 | 0.200 |
| se | | 0.038 | 0.358 | 0.425 | 0.440 | 0.207 |
| t | | −2.128 | 1.436 | −0.11 | −2.106 | 0.967 |
| P | | 0.0336 | 0.1516 | 0.9124 | 0.0355 | 0.3337 |
| SLEDAI_New | R\hat{ }2 | ECR1 | EC4d | EC3d | C3 | C4 |
| coeff | 0.019 | −0.081 | 0.499 | −0.062 | −0.643 | −0.261 |
| se | | 0.038 | 0.360 | 0.425 | 0.547 | 0.269 |
| t | | −2.119 | 1.386 | −0.146 | −1.175 | −0.972 |
| P | | 0.0344 | 0.1660 | 0.8843 | 0.2404 | 0.3314 |

| SLEDAI_New | R\hat{ }2 | ECR1 | EC4d | EC3d | C3 | C4 | dsDNA |
|---|---|---|---|---|---|---|---|
| coeff | 0.020 | −0.080 | 0.480 | −0.071 | −0.634 | −0.243 | 0.186 |
| se | | 0.038 | 0.360 | 0.426 | 0.547 | 0.270 | 0.208 |
| t | | −2.097 | 1.332 | −0.166 | −1.158 | −0.902 | 0.897 |
| P | | 0.0364 | 0.1832 | 0.8679 | 0.2473 | 0.3676 | 0.3702 |

TABLE 19
CB-CAP Biomarkers Plus Traditional Markers for SLEDAI_Tot

| SLEDAI_Tot | R\hat{ }2 | C3 | C4 | dsDNA |
|---|---|---|---|---|
| coeff | 0.159 | −2.215 | −0.753 | 1.932 |
| se | | 0.592 | 0.289 | 0.226 |
| t | | −3.742 | −2.605 | 8.546 |
| P | | 0.0002 | 0.0094 | 7.30E−17 |

| SLEDAI_Tot | R\hat{ }2 | ECR1 | EC4d | EC3d |
|---|---|---|---|---|
| coeff | 0.034 | −0.156 | 0.765 | 0.994 |
| se | | 0.040 | 0.364 | 0.440 |
| t | | −3.911 | 2.105 | 2.259 |
| P | | 0.0001 | 0.0356 | 0.0241 |

| SLEDAI_Tot | R\hat{ }2 | ECR1 | EC4d | EC3d | dsDNA |
|---|---|---|---|---|---|
| coeff | 0.125 | −0.153 | 0.654 | 0.462 | 2.006 |
| se | | 0.042 | 0.397 | 0.471 | 0.228 |
| t | | −3.652 | 1.649 | 0.981 | 8.786 |
| P | | 0.0003 | 0.0996 | 0.3270 | 1.30E−17 |
| SLEDAI_Tot | R\hat{ }2 | ECR1 | EC4d | EC3d | C4 |
| coeff | 0.078 | −0.136 | 0.673 | 0.322 | −1.459 |
| se | | 0.043 | 0.412 | 0.488 | 0.247 |
| t | | −3.134 | 1.635 | 0.66 | −5.897 |
| P | | 0.0018 | 0.1025 | 0.5096 | 5.80E−09 |

| SLEDAI_Tot | R\hat{ }2 | ECR1 | EC4d | EC3d | C4 | dsDNA |
|---|---|---|---|---|---|---|
| coeff | 0.156 | −0.127 | 0.488 | 0.230 | −1.247 | 1.893 |
| se | | 0.042 | 0.395 | 0.467 | 0.238 | 0.228 |
| t | | −3.057 | 1.236 | 0.492 | −5.235 | 8.308 |
| P | | 0.0023 | 0.2168 | 0.6229 | −2.10E−07 | 4.70E−16 |

| SLEDAI_Tot | R\hat{ }2 | ECR1 | EC4d | EC3d | C3 |
|---|---|---|---|---|---|
| coeff | 0.085 | −0.124 | 0.894 | 0.365 | −3.210 |
| se | | 0.043 | 0.408 | 0.485 | 0.501 |
| t | | −2.852 | 2.189 | 0.752 | −6.408 |
| P | | 0.0045 | 0.0289 | 0.4521 | 0.0000 |

| SLEDAI_Tot | R\hat{ }2 | ECR1 | EC4d | EC3d | C3 | dsDNA |
|---|---|---|---|---|---|---|
| coeff | 0.166 | −0.114 | 0.675 | 0.254 | −2.889 | 1.914 |
| se | | 0.042 | 0.391 | 0.463 | 0.480 | 0.226 |
| t | | −2.758 | 1.726 | 0.548 | −6.018 | 8.469 |
| P | | 0.0060 | 0.0848 | 0.5836 | 2.80E−09 | 1.30E−16 |

| SLEDAI_Tot | R\hat{ }2 | ECR1 | EC4d | EC3d | C3 | C4 |
|---|---|---|---|---|---|---|
| coeff | 0.094 | −0.120 | 0.776 | 0.283 | −2.236 | −0.802 |
| se | | 0.043 | 0.409 | 0.484 | 0.622 | 0.306 |
| t | | −2.768 | 1.895 | 0.584 | −3.593 | −2.619 |
| P | | 0.0058 | 0.0585 | 0.5592 | 0.0003 | 0.0090 |

| SLEDAI_Tot | R\hat{ }2 | ECR1 | EC4d | EC3d | C3 | C4 | dsDNA |
|---|---|---|---|---|---|---|---|
| coeff | 0.171 | −0.112 | 0.588 | 0.193 | −2.144 | −0.618 | 1.878 |
| se | | 0.041 | 0.392 | 0.463 | 0.596 | 0.294 | 0.226 |
| t | | −2.692 | 1.498 | 0.417 | −3.6 | −2.104 | 8.307 |
| P | | 0.0073 | 0.1345 | 0.6771 | 0.0003 | 0.0357 | 4.80E−16 |

It is understood that the disclosed invention is not limited to the particular methodology, protocols and materials described as these may vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A method for diagnosing Systemic Lupus Erythematosus (SLE) comprising
determining the level of a first biomarker in a sample from a subject,
wherein if the level of the first biomarker is within a first predetermined level, the subject is diagnosed with SLE,
wherein if the level of the first biomarker is outside the predetermined level, then the level of a second biomarker is determined, and
wherein if the level of the second biomarker is within a second predetermined level, the subject is diagnosed with SLE, and
wherein the first and second biomarker are different and selected from the group consisting of BC4d, EC4d, PC4d and TC4d.

2. The method of claim 1, wherein the subject is negative for the antinuclear antibody test or anti-double stranded DNA test.

3. The method of claim 1, further comprising determining the level of a third biomarker if the level of the second biomarker is outside of the second predetermined level, wherein if the level of the third biomarker is within a third predetermined level, the subject is diagnosed with SLE and wherein the third biomarker is different from the first and second biomarker and is EC4d or ECR1 or a combination thereof.

4. The method of claim 3, wherein the first biomarker is BC4d or TC4d and the second biomarker is PC4d.

5. The method of claim 1, wherein the first biomarker is EC4d and the second biomarker is BC4d.

6. The method of claim 3, wherein the first biomarker is PC4d and the second biomarker is TC4d.

7. The method of claim 1, wherein the first biomarker is BC4d, the second biomarker is PC4d, and wherein the level of TC4d is determined if the level of the second biomarker is within a first predetermined range, and wherein the level of EC4d or ECR1 or both is determined if the level of TC4d is within a second predetermined range.

8. A method for diagnosing Systemic Lupus Erythematosus (SLE) comprising determining the level of EC4d and EC3d in a sample from a subject and determining whether the subject has SLE based on the level of EC4d and EC3d.

9. The method of claim 8, further comprising determining the level of ECR1 and determining whether the subject has SLE based on the level of EC4d, EC3d, and ECR1.

10. The method of claim 8, wherein the subject is negative for the antinuclear antibody test or anti-double stranded DNA test.

11. The method of claim 8 further comprising determining the level of at least one additional biomarker selected from the group consisting of PC4d, TC3d, TC4d, BC3d, and MC4d.

12. A method for facilitating diagnosis of SLE comprising
determining the level of a first biomarker in a sample from a subject,
wherein if the level of the first biomarker is outside a predetermined level, then the level of a second biomarker is determined, and
wherein the first and second biomarker are different and selected from the group consisting of BC4d, EC4d, PC4d and TC4d,
providing the level of the first biomarker and the second biomarker to an entity for diagnosis of SLE.

13. The method of claim 12, further comprising determining the level of a third biomarker if the level of the second biomarker is outside of the second predetermined level and wherein the third biomarker is different from the first and second biomarker and is EC4d or ECR1 or a combination thereof.

14. The method of claim 13, wherein the first biomarker is BC4d or TC4d and the second biomarker is PC4d.

15. The method of claim 12, wherein the first biomarker is EC4d and the second biomarker is BC4d.

16. The method of claim 13, wherein the first biomarker is PC4d and the second biomarker is TC4d.

17. The method of claim 12, wherein the first biomarker is BC4d, the second biomarker is PC4d, and wherein the level of TC4d is determined if the level of the second biomarker is within a first predetermined range, and wherein the level of EC4d or ECR1 or both is determined if the level of TC4d is within a second predetermined range.

18. A combination of tests useful for diagnosing SLE comprising 1) a first test for the level of EC4d, 2) a second test for the level of EC3d and 3) a third test for the level of ECR1.

19. The combination of claim 18, further comprising at least one additional test for the level of a biomarker selected from the group consisting of PC4d, TC3d, TC4d, BC3d, and MC4d.

20. A collection of results in a readable format useful for diagnosing SLE comprising 1) the level of EC4d, 2) the level of EC3d and 3) the level of ECR1.

21. A method of detecting the development of drug-induced lupus erythematosus (DILE) in a subject who is being treated with a pharmacological agent comprising determining the level of at least one biomarker in a sample from the subject and determining whether the subject has DILE based on the level of the at least one biomarker.

22. The method of claim 21, wherein the biomarker is selected from the group consisting of BC4d, EC4d, PC4d, TC4d, ECR1, MC4d, TC3d, BC3d, and combinations thereof.
